(19) United States
(12) Patent Application Publication
(10) Pub. No.: US 2006/0002881 A1
(43) Pub. Date: Jan. 5, 2006

(54) Perfluorocarbon-soluble compounds

(76) Inventors: Ching-An Peng, Arcadia, CA (US); Yu-Chih Hsu, San Diego, CA (US)

Correspondence Address: HOGAN & HARTSON L.L.P., 500 S. GRAND AVENUE, SUITE 1900, LOS ANGELES, CA, 90071-2611, US

(21) Appl. No.: 11/120,519

(22) Filed: May 2, 2005

Related U.S. Application Data
(60) Provisional application No. 60/567,282, filed on Apr. 30, 2004.
(60) Provisional application No. 60/635,161, filed on Dec. 10, 2004.

(51) Int. Cl. A61K 8/64; A61K 8/06

(52) U.S. Cl. 424/70.14; 424/70.24

(57) ABSTRACT

The invention describes syntheses and purifications of several perfluorocarbon-soluble compounds having a general formula R_{F}-L-X or R_{F}-L-X-L-R_{F}, wherein R_{F} is a perfluorocarbon group, X is a hydrophilic moiety, and L is an amide linkage of general structure —CO—NH—. The invention also provides microemulsions comprising a PFC forming an oil phase, water or aqueous solution forming a water phase, and a PEG-based fluorosurfactant with an HFB (hydrophilic groups to fluorophilic groups balance) value from about 7 to about 13. The invention also provides a method of amidification of a fluorocompound. The method involves mixing perfluoroacid chloride of a general formula CₙF₂ₙ₊₁COCl with a compound containing an amine group with a general structure R—NH₂ under reaction conditions sufficient to obtain a product having a general formula CₙF₂ₙ₊₁—CONH—R.

This application claims priority to the U.S. Provisional Patent Application No. 60/567,282, filed on Apr. 30, 2004, and the U.S. Provisional Patent Application No. 60/635,161, filed on Dec. 10, 2004.

### FIELD OF THE INVENTION

The present invention relates to perfluorocarbon (PFC)-soluble fluorinated compounds, methods of their synthesis and their use. The present invention also relates to PFC emulsions and microemulsions prepared with fluorinated surfactants.

### BACKGROUND OF THE INVENTION

Throughout this application, various publications are referred to within parentheses. Disclosures of these publications in their entireties are hereby incorporated by reference into this application to more fully describe the state of the art to which this invention pertains. Full bibliographic citation for these references may be found at the end of this application, preceding the claims.

Highly fluorinated organic compounds and materials, including perfluorocarbon (PFC) compounds, have found multiple applications in the chemical, electronic, nuclear, magnetic media, and aerospace industries, when extreme performance and/or resistance to highly corrosive environments are demanded (Riess, 2001). Perfluorocarbons combine high stability and excellent gas-dissolving capability with extreme chemical and biological inertness, which makes them also desirable for a broad range of biomedical applications (Ceschin, 1985). For example, it has been suggested that PFCs could be used as contrast media; oxygen transport agents or “artificial bloods” in the treatment of heart attack, stroke, and other vascular obstructions; as adjuvants to coronary angioplasty; in drug and gene delivery; in ophthalmology, in retinal repair, replacement of vitreous liquid, and for improving oxygen delivery to the eye; in diagnosis and lubrication; in preserving cell cultures, including sperm preservation; and in cancer radiation treatment and chemotherapy (U.S. Pat. No. 5,502,094; LoNostro, 1999; Krafft, 2001; Lowe, 1998; Riess, 2002).

Among PFCs that are said to be useful in such applications are perfluorodecalin, perfluorotrimethyl bicyclo[3.3.1]nonane, perfluoromethyl adamantane, perfluorodimethyl adamantane, perfluoro-2,2,4,4-tetramethylpentane; 9-12C perfluoro amines, e.g., perfluorotripropyl amine, perfluorotributyl amine, perfluoro-1-azatricyclic amines; bromofluorocarbon compounds, e.g., perfluorooctyl bromide and perfluorooctyl dibromide; F-4-methyl octahydroquinolidizine and perfluoro ethers, including chlorinated polyfluorocyclic ethers, to name just a few (U.S. Pat. No. 5,502,094).

Since PFCs are completely water-insoluble at all temperatures, they need to be converted to a water-based, biocompatible system, such as a PFC-in-water microemulsion before intravenous administration (LeNostro, 1999). PFC-in-water emulsions are desirable for liquid breathing aand artificial blood applications requiring simultaneous transport of mineral salts, metabolites and other compounds of biological importance (Ceschin, 1985). Stable water-in-PFC microemulsions are also desirable in certain applications, including MRI of lungs using partial liquid ventilation (Huang, 2002). Water-in-PFC microemulsions may also be useful for stabilization of water-soluble medications within continuous PFC phase.

Unlike emulsions, which appear cloudy and eventually separate, microemulsions are optically clear, fluid, and thermodynamically stable (Ceschin, 1985; Lawrence, 2000). Water-in-PFC and PFC-in-water microemulsions jointly referred to as “PFC microemulsions” hereinafter) are formed spontaneously by adding a suitable surfactant, frequently combined with a co-surfactant, to a non-miscible mixture of water and PFC (Ceschin, 1985).

Surfactants used to form PFC emulsions and microemulsions must be highly soluble in PFCs. Fluorinated surfactants (or fluorosurfactants, i.e. surfactants with hydrophobic tails comprising fluorocarbon moiety) are a logical choice for emulsifying PFCs as they can provide very low PFC/water interfacial tension (Riess, 2001). Several F-alkylated poloxamer and amine oxide derivatives have been investigated (Riess, 2001). A range of fluorosurfactants with a modular structural design was also synthesized and included molecules with various types of polar heads, including polyols, mono- and disaccharides, amino acids, amine oxides, phosphoramides, lipids, etc. (Riess, 2001). However, a wide use of fluorinated surfactants has been impeded by their high cost (Kraft, 2001).

In addition to being hydrophobic, PFCs are also lipophobic and do not dissolve well in organic liquids. Fluorocarbon/hydrocarbon diblocks, such as perfluoroalkyl alkanes CₘF₂ₘ₊₁(CH₂)ₙH, have amphiphilic character (fluorophilic/lipophilic) and can play a role as part of surfactant system at a PFC/water or PFC/hydrocarbon interface (Riess, 2001). Their solubility in organic liquids have been extensively studied (Rabolt et al., 1984; Russell et al., 1986; Turberg and Brady, 1988). Because of the presence of segregated —CF₂—CH₂— linkage, these compounds are more polar than either fluorocarbons or hydrocarbons. Perfluoroalkyl alkanes have been shown to form miscelles in both hydrocarbons and fluorocarbons (Kraft, 2001). Some of them, given relatively high concentrations, can give rise to gel phase when dissolved in several liquids, including hydrocarbons (Twieg et al., 1985) and mixture of perfluorooctane and isooctane (Ku et al., 1997). Perfluoroalkyl alkanes are prepared by a free-radical-initiated addition of perfluoroalkyl iodide to a terminal olefin (Bargigia et al., 1982; Brace, 1979). The procedures involved are not simple and the yield is relatively low.

Thus, there remains a need for improved methods of syntheses of fluorinated surfactants.

### SUMMARY OF THE INVENTION

As discussed above, PFC is a water-immiscible liquid and poor solvent for drug molecules. In order to overcome the obstacle, one approach is to create a water-in-PFC microemulsion system which can confine the drug or gene of interest within nanoparticles dispersed in the continuous PFC phase with the aid of a fluorosurfactant. However, the reported literature in the area of water-in-PFC microemulsion system is limited. Also, in many medical applications, PFC-in-water microemulsion systems are highly desirable to ensure their biocompatibility. Both systems require surfactants.

Since commercially available surfactants are expensive, not very stable and do not produce stable microemulsions, it is one object of the present invention to provide a stable and inexpensive fluorosurfactants. It is another object of the present invention is to develop simple methods for synthesizing such surfactants. These and other objects are achieved in a fluorosurfactant of the present invention having a general formula R_{F}-L-X or R_{F}-L-X-L-R_{F}, wherein R_{F} is a perfluorocarbon group, X is a hydrophilic moiety, and L is an amide linkage of general structure —CO—NH—. In one embodiment, the hydrophilic moiety is selected from the group consisting of polyols, mono- and polysaccharides, polyethylene glycols (PEGs), amino acids, peptides, alginate, amine oxides, phosphoramides, and their derivatives.

In another aspect, the present invention provides a perfluoroalkanamide of a general structure R_{F}-L-R_{H}, wherein R_{F} is a perfluorocarbon group, L is an amide linkage of general structure —CO—NH—, and R_{H}═CₘH₂ₘ₊₁.

In another aspect, the present invention provides a microemulsion comprising a PFC forming an oil phase, water or aqueous solution forming a water phase, and a PEG-based fluorosurfactant. The flyorosurfactant has an HFB (hydrophilic groups to fluorophilic groups balance) value from about 7 to about 13. The HFB value is calculated as HFB=(mol % hydrophilic group)/5.

In still another aspect, the present invention discloses a method of amidification of a fluorocompound. The method comprises mixing perfluoroacid chloride of a general formula CₙF₂ₙ₊₁COCl with a compound containing an amine group and having a general structure R—NH₂ under reaction conditions sufficient to obtain a product having a general formula CₙF₂ₙ₊₁—CONH—R.

In its final aspect, the present invention provides a method of preparing PFC-in-water or water-in-PFC emulsions comprising:

(a) calculating HFB value of fluorosurfactants using a formula:
HFB=(mol % hydrophilic group)/5;

(b) selecting a candidate fluorosurfactant with HFB value in the range from about 6 to about 13; and

(c) mixing the selected fluorosurfactant with an aqueous solution or water and PFC.

In one embodiment, step (b) further comprises a step of selecting a fluorosurfactant with CₙF₂₊₁ group, which has a molecular weight, structure, or both molecular weight and structure similar to that of the PFC.

The present invention provides a number of advantages over conventional surfactants and methods of their synthesis. In contrast to commercially available fluorosurfactants, such as ZONYL® fluorosurfactants (Du Pont™) of the general formula CₘF₂ₘ₊₁(C₂H₄O)ₙH, the fluorosurfactants of the present invention are advantageously more monodispersed and produce substantially more stable PFC microemulsions, which can be maintained at 25° C. over a period of at least one month. Furthermore, as compared to the perfluoroalkylated PEG surfactants produced by previous researchers, the yields of R_{F}—CONH-PEG surfactants of the present invention are substantially higher (above 95%). In addition to high yield, R_{F}—CONH-PEG surfactants of the present invention are very stable when dissolved in aqueous phase, possibly, due to their sturdy amide linkage (Challis, 1979).

Additionally, the present invention offers the advantages that 1) much enlarged region of water-in-perfluorocarbon microemulsions can be prepared with the aid of perfluorocarbon-soluble surfactants of the present invention; and 2) that the solubilization of pegylated drug (e.g., protein, cytokine) into perfluorocarbon is increased in the present invention through the high perfluorocarbon solubility of fluorosurfactants composed of ethylene oxide chains.

Accordingly, the surfactants and microemulsions of the present invention are well-suited for use in many biomedical and other applications that require stable and inexpensive water-in-PFC and PFC-in-water microemulsions.

### DESCRIPTION OF THE FIGURES

The above-mentioned and other features of this invention and the manner of obtaining them will become more apparent, and will be best understood by reference to the following description, taken in conjunction with the accompanying drawings. These drawings depict only a typical embodiment of the invention and do not therefore limit its scope. They serve to add specificity and detail, in which:

FIG. 1. The FT-IR absorption spectra of synthesized (a) C₇F₁₅COCl, (b) NH2-MPEG_{350,} and (c) C₇F₁₅CONH-MPEG₃₅₀.

FIG. 2. Phase behavior of PFOB/H₂O/C₇F₁₅-MPEG₃₅₀. The five vials represent different phases: (A) PFOB-in-water emulsion, (B) bicontinuous phase, (C) water-in-PFOB emulsion, (D homogeneous PFOB/water emulsion (the volume ratio of PFOB and water is close to unity), and (E) PFC-in-water microemulsion.

FIG. 3. Ternary phase diagram of PFOB/H₂O/C₇F₁₅-MPEG₃₅₀. Various domains were identified: PFOB microemulsion (▪), crystalline gel-like PFOB microemulsion (□), PFOB-in-water emulsion (▴), water-in-PFOB emulsion (●), PFOB emulsion with bicontinuous phase (Δ), and PFOB emulsion with homogeneous phase (◯).

FIG. 4. Ternary phase diagrams with defined points (designated as ▪) of PFOB and ZT85 microemulsions prepared by R_{F}-MPEG₃₅₀, R_{F}-MPEG₅₅₀, and R_{F}-MPEG₇₅₀, respectively.

FIG. 5. FT-IR absorption spectrum of C₆F₁₃—CO—NH—C₁₂H₂₅ fluorosurfactant.

### DETAILED DESCRIPTION OF THE INVENTION

PFCs are hydrocarbons in which most or all of the hydrogen atoms have been replaced with fluorine (Lowe, 1998). PFC microemulsions, including PFC-in-water and water-in-PFC microemulsions, have been considered in a number of biomedical applications due to their high stability and capacity of dissolving oxygen and carbon dioxide combined with excellent stability and biocompatibility (Riess, 2001; Winslow, 2002). Since PFCs are immiscible with aqueous media, surfactants are thereby required to make PFC microemulsions. It has been reported that transparent PFC microemulsion can only be prepared with the aid of fluoroalkylated surfactants due to their capability of lowering the interfacial tension between PFC and water to a near-zero value (Prince, 1967).

The synthesis of poly(oxyethylene) perfluoroalkyl surfactants with the structure of CₘF₂ₘ₊₁CH₂—(OC₂H₄)ₙOH was made by the Williamson-type reaction using fluoroalcohol (CₘF₂ₘ₊₁CH₂OH) and ethylene oxide as the reactants (Selve, 1983; Mathis, 1984). However, this type of reaction results in an uncontrolled polyaddition of ethylene oxide and a broad distribution of oligomers of variable rank n. Thus, the overall yields of fluorosurfactant with m=6 or 7 and n=4-7 were ranged from 10 to 40%. Such low yield is because fluoroalcohol is thermally unstable and difficult to be isolated (Banus, 1951; Lovelace, 1958).

Recently, 70% yield of perfluoroalkylated PEG surfactant with ester linkage was synthesized by reacting perfluorocarbonic acid (CₙF₂ₙ₊₁COOH) with MPEG (LoNosttro, 1999). Although the yield has been improved, the ester bond of R_{F}-PEG is easily hydrolyzed in aqueous solution (Hsu, 2001). ZONYL® nonionic fluorosurfactants (Du Pont™) of the general formula CₘF₂ₘ₊₁(C₂H₄O)ₙH, have been widely used to solubilize perfluorocarbons for the preparation of PFC emulsions (Mathis, 1984). However, due to the polydispersity of this series of surfactants (the values of m and n have a wide distribution), ZONYL® fluorosurfactants couldn't form very stable microemulsion (Schubert, 1994).

Accordingly, in one aspect, the present invention provides a series of novel fluorosurfactants having a general formula R_{F}-L-X, wherein R_{F} is a perfluorocarbon group, L is amide linkage of general structure —CO—NH—, and X is a hydrophilic moiety. The fluorosurfactants of the present invention are advantageously more stable and produce more stable PFC microemulsions.

The perfluorocarbon group R_{F} has the following general formula: CₙF₂ₙ₊₁. In one embodiment, n is in the range from 3 to 17. In one embodiment n is in the range from 7 to 13.

There is no limitation on types of hydrophilic moieties that can be used in the present invention as long as they can be attached to a perfluorocarbon group via an amide linkage. Examples of such hydrophilic moieties include, but are not limited to polyols, mono- and polysaccharides (including chitosan), polyethylene glycols (PEGs), amino acids, peptides, alginate, amine oxides, phosphoramides, and their derivatives. In one embodiment, the hydrophilic moiety comprises a PEG. Because PEG is biocompatible it is particularly suitable hydrophilic moiety for medical applications.

Polyethylene glycols are polymers of ethylene oxide with the generalised formula HO—(CH2—CH2-O)n-H, with ‘n’ indicating the average number of oxyethylene groups. PEGs are typically produced by base-catalyzed ring-opening polymerization of ethylene oxide. The reaction is initiated by adding ethylene oxide to ethylene glycol, with potassium hydroxide as catalyst. This process results in a polydispersed mixture of polyethylene glycol polymers having a molecular weight within a given range of molecular weights. Polyethylene glycols are commercially available in average molecular weights ranging from 200 to 8000 Daltons and, usually, designated by a number indicating the average molecular weight. In one embodiment, an average molecular weight of PEG is from about 200 to about 1000 Daltons.

In another embodiment, the hydrophilic moiety comprises a methoxy-polyethylene glycol (MPEG), also referred to as polyethylene glycol methyl ether. MPEGs are high molecular weight polymers similar in structure and nomenclature to the polyethylene glycols, which can be represented by the generalized formula CH3O—(CH2-CH2-O)n-H, with ‘n’ indicating the average number of oxyethylene groups. MPEGs are commercially available in average molecular weights ranging from 350 to 5000. In one embodiment, MPEGs with the average molecular weight in the range from 350 to 750 are used.

In another aspect, the present invention provides an emulsion comprising a PFC forming an oil phase, an aqueous solution forming a water phase (referred to hereinafter as water), and the fluorsurfactant of the present invention. The amounts of PFC, water, and the fluorosurfactant present in the emulsions of this invention may vary over a range of concentrations. The amount of each ingredient depends on the type of emulsion being prepared (PFC-in-water, water-in-PFC, emulsion, or microemulsion) and relative amounts of the other two components. The actual concentration to produce an acceptable emulsion for any given set of components is easily determined as taught by this invention using the simple techniques of preparing and testing the stability of emulsions at various PFC, water and fluorosurfactant concentrations. In one embodiment, a ternary phase diagram is used to determine the proportions of the fluorosurfactant, PFC, and water to yield a water-in-PFC emulsion, a PFC-in-water emulsion, a water-in-PFC microemulsion, and a PFC-in-water microemulsion.

In one embodiment, the emulsion is a stable clear microemulsion. For the purposes of the present invention, the phrase “stable microemulsion” means that the microemulsion remains clear and does not exhibit phase separation during storage at room temperature. In one embodiment, the microemulsion remains stable for at least one month at 25° C.

The present invention does not impose any limitations on the type of PFC used. But since chemical structure of PFC may have dominant effects on the size of microemulsion domain, the choice of PFC may very between applications depending on the desirable size of microemulsion domain.

PFCs may be straight-chained, branched, or cyclic, or have a combination of such structures. The skeletal chain of the PFC can contain one or more skeletal heteroatoms such as divalent oxygen, a trivalent nitrogen, or a hexavalent sulfur, bonded only to carbon atoms. In one embodiment, the PFC compound has from about 5 to about 12 carbon atoms. U.S. Pat. Nos. 6,297,308; 4,742,050; and 5,502,094, which are incorporated herein by reference, describe various PFC compound that has been used in various applications. Such PFCs include, but are not limited to: perfluorodecalin, perfluoroindane, perfluorotrimethyl bicyclo[3.3.1]nonane, perfluoromethyl adamantane, perfluoro-2,2,4,4-tetramethylpentane; 9-12C perfluoro amines, e.g., perfluorotripropyl amine, perfluorotributyl amine, perfluoro-1-azatricyclic amines; bromofluorocarbon compounds, e.g., perfluorooctyl bromide and perfluorooctyl dibromide; F-4-methyl octahydroquinolidizine and perfluoro ethers, including chlorinated polyfluorocyclic ethers, perfluoropolyether, perfluoro-4-methylmorpholine, perfluorotriethylamine, perfluoro-2-ethyltetrahydrofuran, perfluoro-2-butyltetrahydrofuran, perfluoropentane, perfluoro(2-methylpentane), perfluorohexane, perfluoro-4-isopropylmorpholine, perfluorodibutyl ether, perfluoroheptane, perfluorooctane, perfluorotripropylamine, perfluorononane, perfluorotributylamine, perfluorodihexyl ether, perfluoro[2-(diethylamino)ethyl-2-(N-morpholino)ethyl]ether, n-perfluorotetradecahydrophenanthrene, and mixtures thereof. These and other PFCs could be used with surfactants of the present invention.

In one embodiment, the PFC is perfluoroctyl bromide (C₈F₁₇Br, PFOB) or perfluoropolyether (HF₂C—(OC₂F₄)ₙ—(OCF₂)ₘ—OCF₂H, Galden®ZT85, PFPE). PFOB is stable, well tolerated and can be rapidly eliminated from the body. It is not only a universal contrast agent applicable for X-ray, CT scanning, ultrasound and magnetic resonance imaging but also a therapeutic oxygen carrier (Long, 1988). PFPE is a well-known fluid with the presence of the most electronegative atoms (O F) bonded to C—C groups along the molecular chain assuring highly chemical and thermal stability. As a result, PFPE fluids offer a unique combination of performance as lubricants, which are highly reliable even under very harsh conditions (Caporiccio, 1986). Moreover, the PFPE-based materials show a potential for use in the development of biomaterials in the ocular, vascular, and orthopedic areas (Johnson, 1999).

The water phase may be water alone or an aqueous solution containing buffering agents, salts, or other reagents that make the resulting solution physiologically acceptable. For example, a saline solution isotonic with blood such as Ringer's or Tyrode's solution may be used. The aqueous solution may also contain a therapeutic agent, including, but not limited to, water-soluble drugs, gene-containing compositions, and nutrients.

Other materials conventionally employed in pharmaceutical preparations and known to the skilled formulator may also be added to the emulsions of the present invention. These include viscosity modifiers, stabilizers (against degradation due to freezing or contamination, for example), anti-freeze agents, diluents, encoding agents, and the like. Among such additives may be mentioned glycerin, dimethylsulfoxide (“DMSO”), various gelatins both natural and synthetic, and polyols such as sorbitol.

In order to obtain a PFC microemulsion without use of a co-surfactant, it is believed that the surfactant should be both sufficiently hydrophilic and fluorophilic (Ceschin, 1985). Those skilled in the art have been using a hydrophile-lipophile balance (HLB) formula deduced by Griffin (Griffin, 1954) to predict which nonionic surfactants surfactants could be used to form microemulsions (Myers, 1988; Selve, 1983). It is presumend that surfactants with 3-6 HLB values are favorable for the formation of water-in-oil microemulsions and HLB values of 8-18 are preferred for the formation of oil-in-water microemulsions (Lawrence, 2000).

By analogy with HLB, the inventors developed an empirical formula for calculating HFB (hydrophilic groups to fluorophilic groups balance) value of PEG-containing fluorosurfactants:
HFB=(mol % hydrophilic group)/5.

The inventors unexpectedly discovered that PEG-based fluorosurfactants having HFB value in the range from 7 to 13 are capable of stabilizing PFC microemulsion without an aid of a secondary surfactant.

Accordingly, in another aspect, the present invention provides a microemulsion comprising a PFC compound, water, and a PEG-based fluorosurfactant, wherein the fluorosurfactant has hydrophilic groups to fluorophilic groups balance (HFB) value in the range from 7 to 13. In one embodiment, the fluorosurfactant is selected from a group consisting of C₇F₁₅CONH-PEG₃₅₀-OCH₃ (HFB=8.8), C₇F₁₅CONH-PEG₅₅₀-OCH₃(HFB=11.2), and C₇F₁₅CONH-PEG₇₅₀-OCH ₃(HFB=12.7). In another embodiment, the fluorosurfactant comprises MPEG having a molecular weight from about 350 to about 750 Daltons.

In another embodiment, the fluorosurfactant comprises a first perfluorocarbon group and the PFC comprises a second perfluorocarbon group and the fluorosurfactant is selected in such a way that the structure of the first and the second perfluorocarbon groups have a similar structure. Those skilled in the art would know how to compare structure of two perfluorocarbon groups. For example, the length and shape (straight, branched, cyclical, etc.) of the skeletal chain, presence and quantity of skeletal heteroatoms, molecular weight and other chemical, physical, and structural characteristics may be evaluated. In another embodiment, the first and the second perfluorocarbon groups have a similar molecular weight. However, emulsions and microemulsions, in which perfluorocarbon groups of the PFC and the fluorosurfactant are different in structure and molecular weight are also within the scope of the present invention.

In another aspect, the present invention provides a method of high yield synthesis of amidified fluorocompounds, in general, and perfluoroalkylated PEG surfactants with amide linkage and perfluoroalkanamides, in particular.

As discussed above, fluorosurfactants that are both sufficiently hydrophilic and fluorophilic are desirable when preparing stable microemulsions. A hydrophilic moiety may be chemically grafted to a perfluorocarbon group to form an end-functionized perfluoroalkylated surfactant. In one embodiment, poly(ethylene glycol) was selected as the hydrophilic moiety due to its biocompatible characteristics for medical application. The following general reaction schemes A and B provide examples of four end-functionized perfluoroalkylated poly(ethylene glycol) surfactants.

A. Esterification

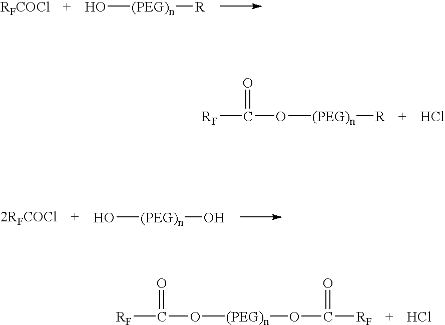

where, R═H or hydrocarbon compounds, R_{F} represents the perfluorocarbon group

B. Amidification

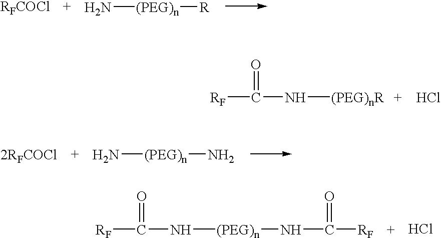

where, R═H or hydrocarbons, R_{F} represents the perfluorocarbon group

The following reaction Schemes I and II provide further details of amidification method of the present invention. Scheme I schematically demonstrates synthesis of perfluoroalkylated PEG-based surfactant. Scheme II schematically shows synthesis of perfluoroalkanamides.

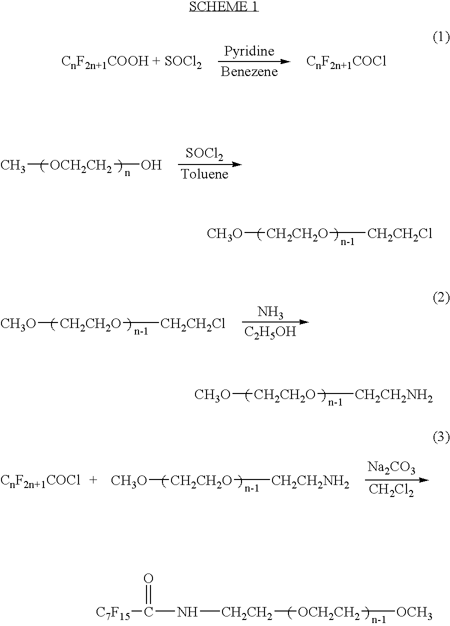

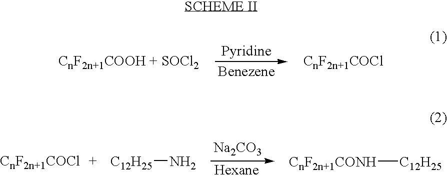

More generally, the present invention provides a method of amidification of a fluorocompound. The method comprises mixing perfluoroacid chloride of a general formula CₙF₂ₙ₊₁COCl with a compound containing an amino group and having a general structure R—NH₂ under reaction conditions sufficient to obtain a product having a general formula CₙF₂ₙ₊₁—CONH—R. In some embodiments, such as those described in Examples 3 and 9, perfluoroacid chloride is dissolved in hexane to obtain a first mixture prior to mixing perfluoroacid chloride with the amino-modified compound. In embodiment of Example 3, dichloromethane is mixed with the amino-modified compound to form a second mixture prior to combining with the first reaction mixture.

For the purposes of the present invention, the reaction conditions are sufficient if they facilitate production of the product. Such reaction conditions include, but are not limited to, incubation time and temperature, manner in which ingredients are combined (pouring, drop-by-drop addition, etc.), and concentrations of the ingredients. The selection of suitable conditions are well within the scope of the skills of those skilled in the art and depend on the desired product yield and a specific type of amino-modified compound used.

For example, in one embodiment of the present invention, the reaction conditions comprise incubation for 6 hours at 4° C. on ice (Example 3), while in another embodiment the reaction conditions comprise incubation at room temperature for 30 minutes, followed by incubation at 60° C. for 6 hours. In another embodiment, the reaction conditions comprise adding a mixture of perfluoroacid chloride and hexane by drops into a flask containing dichloromethane and an amino-modified compound. In another embodiment, the reaction conditions comprise combining the compound containing an amino group and perfluoroacid chloride in the 1:1.1 molar ratio. Based on the teachings of the instant specification and their knowledge of basic chemistry, those skilled in the art would be able to select other suitable reaction conditions without undue experimentation.

### EXAMPLES

Materials

Methoxy-polyethylene glycols (MPEG) with molecular weight of 350, 550 and 750 were obtained from Union Carbide (Danbury, Conn.), perfluorooctanoic acid (C₇F₁₅COOH), perfluorododecanoic acid (C₁₁F₂₃COOH) and perfluorotetradecanoic acid (C₁₃F₂₇COOH) were from Aldrich (Milwaukee, Wis.), perfluoroctyl bromide (PFOB) from Chem-surf (Sebastopol, Calif. ) and polyfluoropolyether Galden®ZT85 from Solvay Solexis, Inc. (Thorofare, N.J.). Perfluoroheptanoic acid (C₆F₁₃COOH, 97.6%), perfluorooctanoic acid (C₇F₁₅COOH, 98%) and perfluoronanoic acid (C₈F₁₇COOH, 98%) were obtained from P & M Company (Russia). Dodecylamine (C₁₂H₂₅—NH₂, 98%) and octylamine (C₈H₁₇—NH_{2,} 98%) were purchased from Fluka (USA). CF-76 was received from 3M (USA). Other chemicals if not specified were purchased from Sigma or Aldrich. All of these materials were used as received without further purification.

### EXAMPLE I

Synthesis of Perfluoroacid Chloride

Step (1) of the Scheme I and Scheme II

One mole ratio of a perfluorocarbonic acid (C₆F₁₃COOH, C₇F₁₅COOH, C₈F₁₇COOH, C₁₁F₂₃COOH or C₁₃F₂₇COOH) was first dissolved in 100 ml of dry benzene under a nitrogen atmosphere. Then, 2 mole ratio of thionyl chloride (SOCl₂) and 0.5 mole ratio of pyridine were added. The mixture was placed in a 250 ml 3-necked round bottom flask equipped with a reflux condenser, a drying tube, a stirring bar and a N₂-inlet. The reaction commenced immediately with an appearance of fumes due to the evolution of HC1. The contents were continuously refluxed for 6 hours at 60° C. The traces of excess of thionyl chloride and reaction solvent were evaporated in vacuum at 80° C. The residue was used directly in the subsequent amidation with amino-MPEG (Example 3), dodecylamine (C₁₂H₂₅—NH₂) or octylamine (C₈H₁₇—NH₂) (Example 9).

### EXAMPLE 2

Calculation of HFB Values of Fluorosurfactants

The HFB values of PEG-based fluorosurfactants were calculated using the following formula:
HFB=(mol % hydrophilic group)/5.

A series of perfluoroalkylated MPEG surfactants, listed in Table I, were thereby synthesized to examine their potency of making PFC microemulsion and their HFB values were calculated.

TABLE 1
Chemical formula of synthesized R_{F}-MPEG fluorosurfactants
with calculated HFB values

| Structure of fluorosurfactant | HFB |
|---|---|
| C₇F₁₅CONH-PEG₃₅₀-OCH₃ | 8.8 |
| C₇F₁₅CONH-PEG₅₅₀-OCH₃ | 11.2 |
| C₇F₁₅CONH-PEG₇₅₀-OCH₃ | 12.7 |
| C₁₁F₂₃CONH-PEG₃₅₀-OCH₃ | 7.3 |
| C₁₁F₂₃CONH-PEG₅₅₀-OCH₃ | 9.4 |
| C₁₃F₂₇CONH-PEG₃₅₀-OCH₃ | 6.7 |
| C₁₃F₂₇CONH-PEG₇₅₀-OCH₃ | 10.2 |

### EXAMPLE 3

Synthesis of Perfluoroalkylated PEG Surfactant

Steps (2) and (3) of the Scheme I

Terminal hydroxyl group of MPEG was converted into amino group by a two-step polymer analogous reaction (Step (2) of the Scheme I). MPEG-OH[(CH₃—(OCH₂CH₂)ₙ—OH] was dissolved in toluene and dried by azeotropic distillation. Then, a few drops of pyridine were added into 250 ml 3-necked round bottom flask which contained 10 g MPEG-OH and 150 ml toluene. Thionyl chloride, freshly distilled from quinoline, was added drop-wise into the reaction flask for one hour. The molar ratio of MPEG-OH to SOCl₂ was 2:3. The mixture was heated at 65° C. for 4 hours, and then cooled to room temperature. After filtering pyridine hydrochloride and evaporating toluene containing excess of thionyl chloride in vacuum at 80° C., the residue was dissolved in CH₂Cl₂, dried over anhydrous K₂CO₃ and filtered. The product of CH₃O—(CH₂CH₂O)ₙ₋₁—CH₂CH₂Cl was then precipitated in diethyl ether. Subsequently, CH₃O—(CH₂CH₂O)ₙ₋₁—CH₂CH₂Cl was dissolved in ethanol solution saturated with excess of ammonia, heated up to 70° C., and maintain high pressure in the reaction flask for 24 hours. The resulting product, MPEG-NH₂(CH₃O—(CH₂CH₂O)ₙ₋₁—CH₂CH₂NH₂) was precipitated in diethyl ether and dried in vacuum.

Synthesis of perfluoroalkylated MPEG surfactant was carried out according to the step (3) of the Scheme I. The molar ratio of amino-MPEG to perfluoroacid chloride in reaction mixture was 1:1.1. Specifically, a 250 ml two-necked round-bottomed flask equipped with a reflux condenser used for the synthesis was immersed in a trough containing ice to assure the active ester reaction maintained at 4° C. First, the amount of 20 ml CH₂Cl₂ containing 0.05 mole of hydrophilic reagent (i.e., poly(ethylene glycol)) was poured into the flask and mixed by a magnetic stirrer. In order to remove the hydrochloride generated from the ester reaction, 0.05 mole of Na₂CO₃ was added in advance into the solution for absorbing HCl.

Then, 0.05 mole of hydrophobic reagent (i.e., R_{F}COCl) was added drop-wise through a dropping funnel into the flask. To prevent any oxidization of reactants and products, nitrogen gas was injected into the reaction flask to purge out oxygen during the synthesis. It took approximately one hour to have all of hydrophobic reagent added gradually into the flask. After that, the reaction was continued for another 5 hours. The reaction was run total of six hours on ice at 4° C. The product mixture was filtered through a filter paper to remove Na₂CO₃ particles. The filtered solution was transferred to a rotary evaporator heated at 60° C. under vacuum to remove CH₂Cl₂. After cooling down, the left product was the perfluoroalkylated poly(ethylene glycol) surfactant.

### EXAMPLE 4

Characterization of Perfluoroalkylated PEG Surfactant

The molecular weight of the synthesized surfactants was determined by gel permeation chromatography (GPC). The GPC system consisted of a Shodex OHPak KB-806M column (Showa Denko, Tokyo, Japan), a Water 410 differential refractometer (Waters Associates Inc., Milford, Mass.), and a Waters U6-K multisolvent delivery system (Waters Associates Inc., Milford, Mass.) which pumped the mobile phase through the column. The column temperature was maintained at 40° C. The mobile phase was tetrahydrofuran (THF) and the flow rate was 1.00 mL/min. The synthesized fluorosurfactant was dissolved in THF, and filtered through a 0.2 μm syringe filter (Gelman, Ann Arbor, Mich.) prior to being injected into the system. Monodisperse polyethylene glycol samples (Waters Associates Inc., Milford, Mass.) were used as the calibration standards. Fourier Transform Infrared (FT-IR, Nicolet Magna-IR 860 spectrometer in transmission mode, with a pure silicon wafer as a reference, scan number 100, resolution 4 cm⁻¹) was used to detect the amide (—CO—NH—) linkage of the synthesized perfluoroalkylated MPEG surfactants. A wedged silicon wafer (Harrick Scientific Corp, state) was used because the entered light can be reflected within the Si for several times before it leave. Since the light can interact with the coated material for several times, the signal will be greatly enhanced.

### EXAMPLE 5

Analysis of Synthesized Fluorosurfactants

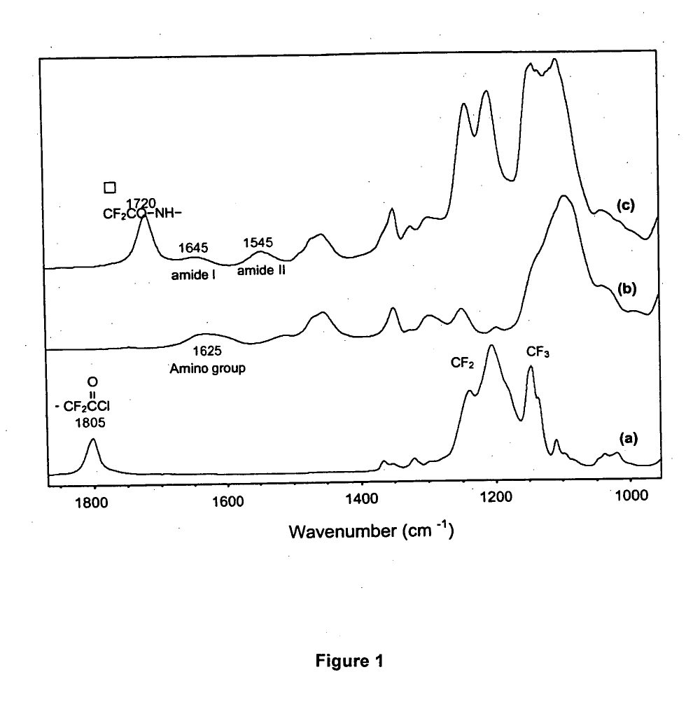

In FIG. 1, the bottom spectrum (a) is the FT-IR measurement of synthesized perfluoroacid chlorides (C₇F₁₅COCl). The peak absorption of C—F bond stretch ranged from 1150 to 1250 cm⁻¹ and the stretching vibration of —CF₂COCl group was detected at 1805 cm⁻¹. The IR spectra of C₁₁F₂₃COCl and C₁₃F₂₇COCl were similar to the one of C₇F₁₅COCl and not shown here. The spectrum of synthesized amino-MPEG350is labeled as (b). The —NH₂ group peak absorption of amino-MPEG350was detected around 1625 cm⁻¹. The spectrum of synthesized perfluoroalkylated MPEG surfactant (c) shows the N—H bending vibrations of amide I and II at 1645 cm⁻¹ and 1545 cm⁻¹, respectively. The adsorption peaked at 1720 cm⁻¹ representing the carbonyl stretching vibration of amide linkage (—CONH—). The purity and yield of synthesized fluorosurfactants were all up to 95% (chromatograms not shown here).

### EXAMPLE 6

Phase Behavior of the Mixture of PFC/Water/Fluorosurfactant

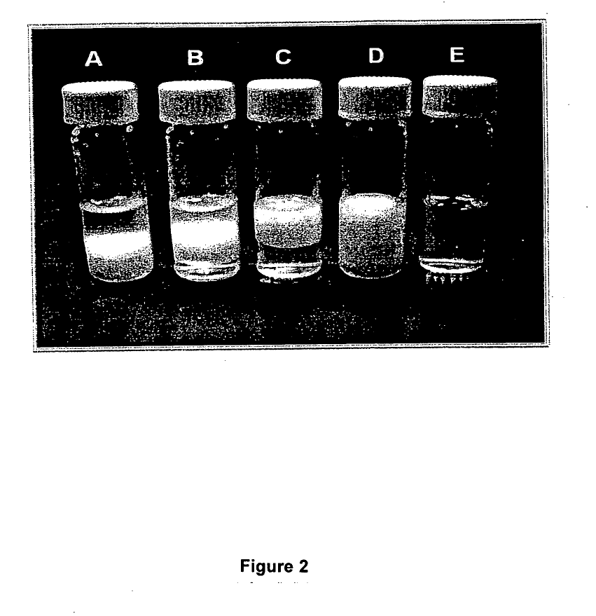

The formation of PFC-in-water emulsion requires high energy to disperse PFC as surfactant-stabilized microdroplets in aqueous phase; while, for thermodynamic stable PFC microemulsion, two immiscible liquid phases with the aid of a pertinent fluorosurfactant can turn into an optically isotropic solution simply by gentle mixing. Given in FIG. 2, different phase behaviors were created by titering various concentrations of C₇F₁₅CONH-MPEG₃₅₀ surfactant dissolved in water solution with different amounts of PFOB liquid. Since the specific density of PFOB is 1.94 which is larger than the water density, the milky PFOB-in-water emulsion settled down on the bottom of the vial (FIG. 2A).

The bicontinuous middle phase given as FIG. 2B indicated excess volume of water and PFOB along with small amount of fluorosurfactant were presented in the vial. When the volume of added PFOB was more than the volume of water, water-in-PFOB emulsion was revealed on the upper with the milky appearance (FIG. 2C). The formation of one phase PFOB-in-water emulsion shown in FIG. 2D occurred as the volume ratio of PFOB and water reached unity. PFC-in-water microemulsion with total transparent appearance (see FIG. 2E) was attained when the composition of PFC, water, and fluorosurfactant were in the right proportions.

### EXAMPLE 7

Setup of Phase Diagram

Aqueous solutions containing given quantities of fluorosurfactant were progressively added with PFC and placed in a thermostat water bath set at 25° C. After each addition, the aspect of the solution was recorded, along with number, nature, and approximate volumes of phases. Mixed solutions were left for one month before observation was completed. To build the feasibility zone, the resulting PFC microemulsion was defined being transparent appearance. Ternary phase diagrams were then plotted to distinguish the boundaries between different phases and used to quantitate the proportion of three components (water, fluorosurfactant, and PFC) forming PFC microemulsion.

### EXAMPLE 8

Phase Diagram of PFC Microemulsion

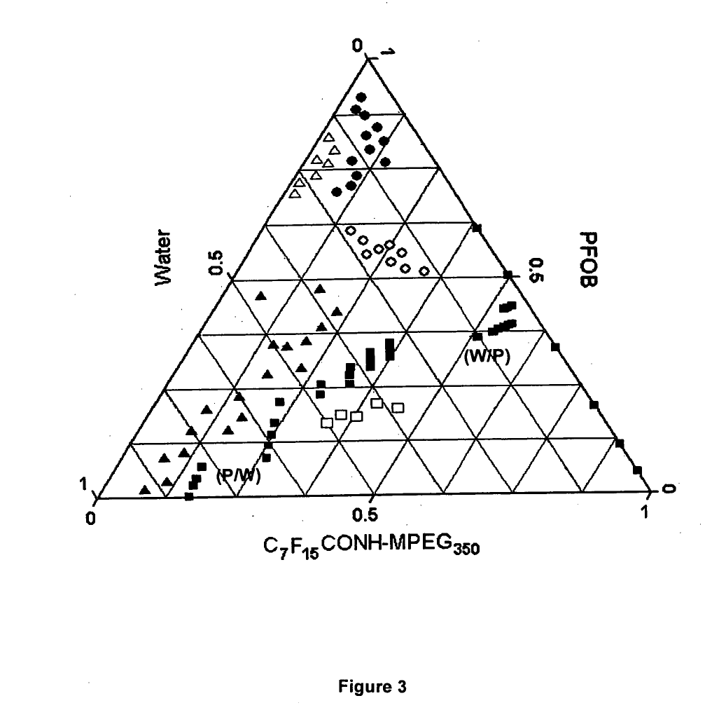

The ternary phase diagram given in FIG. 3 was determined by the weight fraction of water, PFOB, and R_{F}-MPEG₃₅₀. The PFOB microemulsion domain was determined to be the area under the test points symbolized as ▪. Symbols of □ stand for the PFOB microemulsion having crystalline gel-like feature. The PFOB-in-water emulsion represented by the symbols of ▴ has the phase illustrated in FIG. 2A. The ● symbols indicate the compositions that form water-in-PFOB emulsion shown as FIG. 2C. Symbols ◯ and Δ represent the regions of one phase PFOB emulsion (shown as FIG. 2D) and bicontinuous phase of PFOB emulsion shown as FIG. 2b), respectively. The ▪ symbols near the left vortex region of FIG. 3 (designated as P/W) represent the domain of PFOB-in-water microemulsion where water is the continuous phase. The domain of water-in-PFOB microemulsion with the dispersed phase being water (designated as W/P) is located near the right vortex region with plentiful amount of fluorosurfactant. For the region containing homogeneous phase of PFOB emulsion (illustrated by the symbols of ◯), the volume ratio of PFOB to water ranged from 0.9 to 1.3.

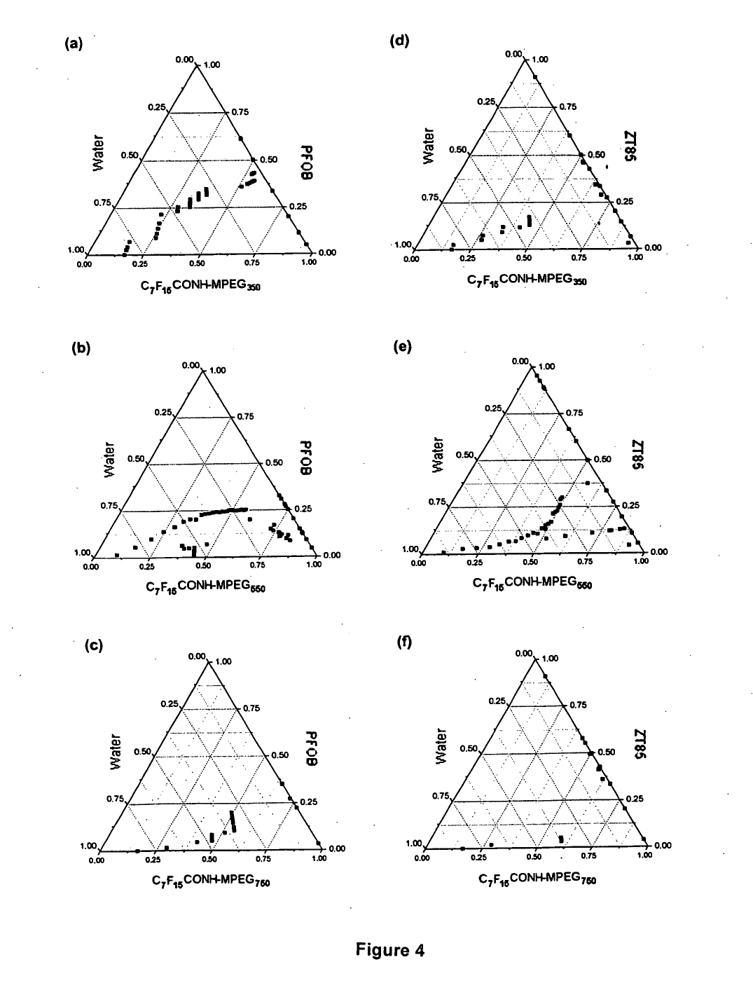

In FIG. 4, PFOB microemulsion domains determined in a series of ternary phase diagrams were established from two PFCs (PFOB and ZT85) in combination with three C₇F₁₅-MPEG surfactants with MPEG MW of 350, 550, and 750. For both PFCs, the lower MW of MPEG used to synthesize R_{F}-MPEG, the larger size of microemulsion domain was obtained. For the rest of fluorosurfactants given in Table I (i.e., R_{F} moiety=C₁₁F₂₃, C₁₃F₂₇), no transparent PFC microemulsion was detected from established ternary diagrams.

### DISCUSSION

The HFB values of R_{F}-MPEG surfactants used to gain PFC microemulsion shown in FIG. 4 were calculated as 8.8, 11.2 and 12.7, respectively. From the domains of PFC microemulsions established in ternary phase diagrams, it appears that the lower the HFB value, the larger the zone of PFC microemulsion can form. In addition, since it is also noticed that the solubility of C₇F₁₅-MPEG in PFOB or ZT85 decreased with the increment of MW of MPEG (i.e., C₇F₁₅-MPEG₃₅₀>C₇F₁₅-MPEG₅₅₀>C₇F₁₅-MPEG₇₅₀), this factor may also play an important role on the observation that C₇F₁₅-MPEG₃₅₀ provides the largest PFC microemulsion region. In comparison of FIG. 4(a)-(c) with (d)-(f), the domains of PFOB microemulsion were much extended than those of ZT-85 microemulsion.

While not wanting to be bound by theory, it has been observed that aside from the effects of HFB value and solubility of R_{F}-MPEG on the formation of PFC microemulsion, perfluorocarbon group (C₈F₁₇—) of PFOB has much similar molecular bonding structure with the perfluoroalkylated moiety (C₇F₁₅—) of PEG-based surfactants than the perfluorocarbon group of ZT-85 has. That may be the reason why PFOB microemulsion regions were broader than the corresponding ZT-85 microemulsion regions. Moreover, since neither C₁₁F₂₃-MPEG nor C₁₃F₂₇-MPEG fluorosurfactants could be used to form PFOB or ZT85 microemulsion, it may be necessary to select a surfactant such that the molecular weight of its fluorophilic group is close to that of PFC (MW of PFOB and ZT-85 is 499 and 353 g/mole, respectively), in order to form a PFC microemulsion. However, emulsions, in which perfluorocarbon groups of the PFC and the fluorosurfactant are different in structure and molecular weight are also within the scope of the present invention.

### EXAMPLE 9

Synthesis of Perfluoroalkanamides

Step (2) of the Scheme II

Synthesis of perfluoroalkanamide-type surfactants was done by the following steps. First, perfluorocarbonic acid was converted to perfluoroacid chloride (step (1)). Then, C₁₂H₂₅—NH₂ (or C₈H₁₇—NH₂) dissolved in hexane with few drops of pyridine was reacted with perfluoroacid chloride (CₙF₂ₙ₊₁COCl, n=6-8) in the presence of Na₂CO₃. The molar ratio of C₁₂H₂₅—NH₂ (or C₈H₁₇—NH₂) to perfluoroacid chloride in reaction mixture was 1:1.1. The mixture was placed in a 3-necked round bottom flask, a stirring bar and a N₂-inlet. T he reaction was run for 30 min at room temperature. After 30-min reaction, the temperature was raised to 60° C. with a reflux condenser for 6 hours. The reacted mixture was filtered from Na₂CO₃ and dried in vacuum. The residue was resolved in acetone then dried in vacuum. The final product CₙF₂ₙ₊₁—CONH—C₁₂H₂₅ (or CₙF₂ₙ₊₁—CONH—C₈H₁₇)fluorosurfactant through amide linkage was washed by de-ionized water twice then dried in oven at 40° C.

### EXAMPLE 10

FT-IR Analysis

Fourier Transform Infrared (FT-IR, Nicolet Magna-IR 860 spectrometer in transmission mode, with a pure silicon wafer as a reference, scan number 100, resolution 4 cm⁻¹) was used to detect the amide (—CO—NH—) linkage of the synthesized perfluoroalkanamides. A wedged silicon wafer (Harrick Scientific Corp, state) was used because the entered light can be reflected within the Si for several times before it leave. Since the light can interact with the coated material for several times, the signal will be greatly enhanced.

### EXAMPLE 11

Characterization of Perfluoroalkanamides

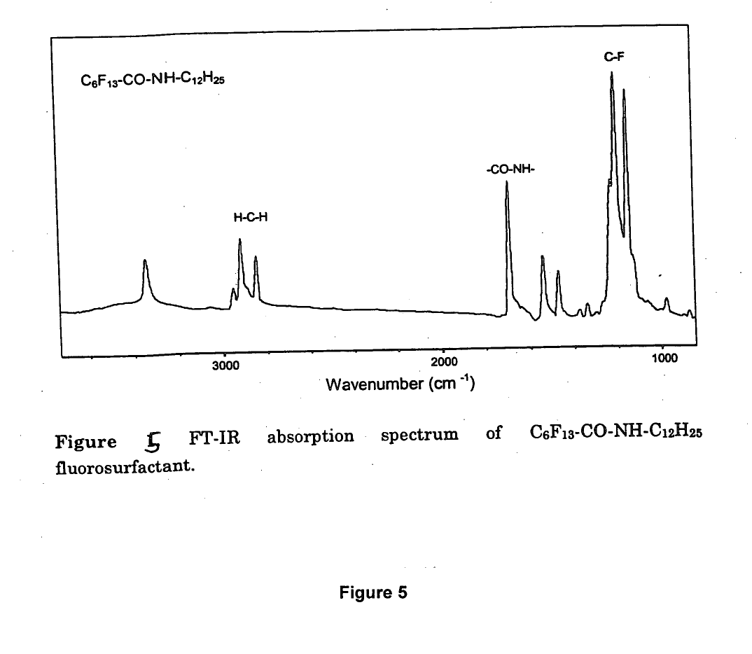

FIG. 5 illustrates the spectrum of synthesized perfluoroalkanamide (C₆F₁₃—CO—NH—C₁₂H₂₅). The N—H bending vibrations of amide I and II were peaked at 1645 cm⁻¹ and 1540 cm⁻¹, respectively. The peak absorption of C—F bond stretch ranged from 1150˜1250 cm⁻¹. The characteristic absorption peaks of H—C—H are located at 2800˜2950 cm⁻¹ and 1470 cm⁻¹. The adsorption peaked at around 1700 cm⁻¹ representing the carbonyl stretching vibration of amide linkage (—CONH—).

### EXAMPLE 12

Solvent Solubility

The solubilities of synthesized perfluoroalkanamides in various solvents under different temperatures are shown in Table 1. It is clear from the observation that perfluoroalkanamides of the present invention can easily form micelles in acetone at room temperature. With shorter perfluoroalkyl group (i.e., C₆F₁₃—), perfluoroalkanamide can dissolve into all tested hydrocarbon solvents at room temperature, but precipitate in fluorocarbon (i.e., CF-76).

Although not wanting to be bound by a theory, the inventors believe that the solubility of perfluoroalkanamides in CF76 increases when the chain length of perfluoroalkyl group increases and the chain length of alkyl group decreases. This theory is consistent with the observation of solubility of C₈F₁₇CONH—C₈H₁₇ in CF76 at room temperature.

TABLE 1

| | C₆F₁₃CONHC₁₂H₂₅ | C₇F₁₅CONHC₁₂H₂₅ | C₈F₁₇CONHC₁₂H₂₅ | C₈F₁₇CONHC₈H₁₇ |
|---|---|---|---|---|
| | (3% w/w) | (3% w/w) | (3% w/w) | (3% w/w) |
| Acetone | S | S | S | S |
| Ethanol | S | @ RT → P > | @ RT → P > | S |
| | | 40° C. → S | 40° C. → S | |
| Hexane | S | @ RT → P > | @ RT → P > | S |
| | | 30° C. → S | 30° C. → S | |
| CF-76 (3 M) | @ RT → P > | @ RT → P > | @ RT → P > | S |
| | 40° C. → S | 40° C. → S | 40° C. → S | |

* S: complete dissolve in the solvent at room temperature; P: precipitation in the solvent; RT: room temperature

The present invention may be embodied in other specific forms without departing from its essential characteristics. The described embodiments are to be considered in all respects only as illustrative and not as restrictive. The scope of the invention is, therefore, indicated by the appended claims rather than by the foregoing description. It is intended that the present invention cover modifications and variations of this invention that come within the scope of the appended claims and their equivalents.

### REFERENCES

1. J. Banus, H. J. Emeleus and R. N. Haszeldine. The heterolytic fission of the carbon-iodine bond in trifluoroiodomethane. J. Chem. Soc., Abstracts:60-64 (1951).

2. Bargigia, GA, Caporiccio, G, Pianca, M. J. Fluorine Chem. 1982, 19, 403.

3. Brace, NO. J. Org. Chem. 1979, 44, 212.

4. G. Caporiccio. Perfluoropolyether fluids: properties and applications. Journal of Fluorine Chemistry 33(1-4):314-20 (1986).

5. C. Ceschin, J. Roques, M. C. Malet-Martino and A. Lattes. Fluorocarbon microemulsions. J. Chem. Tech. Biotechnol. 35A:73-82 (1985).

6. B. C. Challis and J. A. Challis. In comprehensive organic chemistry; The synthesis and reactions of organic compounds. Pergamon Press, 1979, vol 2, p957.

7. Z. Cui and R. J. Mumper. Plasmid DNA-entrapped nanoparticles engineered from microemulsion precursors: in vitro and in vivo evaluation. Bioconjugate chemistry 13(6):1319-27 (2002).

8. W. C. Griffin. Calculation of HLB Values of Non-Ionic Surfactants. J. Soc. Cosmet. Chem. 5:249-256 (1954).

9. Y.-C. Hsu, M. Acuna, S. M Tahara and C.-A. Peng. Reduced Phagocytosis of Colloidal Carriers Using Soluble CD47. Pharmaceut. Res. 20:1539-1542(2003).

10. Y.-C. Hsu and C.-A. Peng. Diminution of phagocytosed perfluorocarbon emulsions using perfluoroalkylated polyethylene glycol surfactant. Biochem. Biophy. Res. Comm. 283:776-781 (2001).

11. M. Q. Huang, Q. Ye, D. S. Williams, C. Ho. MRI of lungs using partial liquid ventilation with water-in-perfluorocarbon emulsions. Magnetic Resonance in Medicine, 48(3): 487-492 (2002).

12. M. Hubler, J. E. Souders, E. D. Shade, N. L. Polissar, C. Schimmel and M. P. Hlastala. Effects of vaporized perfluorocarbon on pulmonary blood flow and ventilation/perfusion distribution in a model of acute respiratory distress syndrome. Anesthesiology 95(6):1414-1421 (2001).

13. G. Johnson, G. F. Meijs, B. G. Laycock, M. G. Griffith, H. Chaouk, and J. G. Steele. Cell interactions with perfluoropolyether-based network copolymers. J Biomat Sci-Polym E 10 (2): 217-233 (1999).

14. Kissa, E. Fluorinated Surfactants and Repellents. 2^{nd} Edition. Marcel Dekker Inc. New York, 2001.

15. J. Klier, C. J. Tucker, T. H. Kalantar and D. P. Green. Properties and applications of microemulsion. Adv. Mater. 12:1751-1757 (2000).

16. M. P. Krafft. Fluorocarbons and fluorinated amphiphiles in drug delivery and biomedical research. Adv. Drug Delivery Rev. 47:209-228 (2001).

17. Ku, C Y, LoNostro, P, Chen, S H, J. Phys. Chem. B 1997, 101, 908.

18. M. J. Lawrence and G. D. Rees. Microemulsion-based media as novel drug delivery systems. Adv. Drug Delivery Rev. 45:89-121 (2000).

19. D. M. Long, D C. Long, R. F. Mattrey, R. A. Long, A. R. Burgan, W. C. Herrick and D. F. Shellhamer. An overview of perflurooctylbromide—application as a synthetic oxygen carrier and imaging agent for x-ray, ultrasound and nuclear magnetic resonance. Biomaterials, Artificial Cells, and Artificial Organs 16(1-3):411-420 (1988).

20. P. LoNosttro, S.-M. Choi, C.-Y. Ku and S.-H. Chen. Fluorinated Microemulsion: A study of the phase behavior and structure. J. Phys. Chem. 103:5347-5352 (1999).

21. L. M. Lovelace, D. A. Rausch and W. Postelnek. Aliphatic fluorine compounds. Reinhold, N.Y., 1958, p 137.

22. K. C. Lowe, M. R. Davey and J. B. Power. Perfluorochemicals: their applications and benefits to cell culture. Tibtech 16:272-277 (1998).

23. G. Mathis, P. Leempoel, J. C. Ravey, C. Selve and J. J. Delpuech. A Novel Class of Nonionic Microemulsions: Fluorocarbons in Aqueous Solutions of Fluorinated Poly(oxyethylene) Surfactants, J. Am. Chem. Soc. 106:6162-6167 (1984).

24. D Myers. Surfactant science and technology. Publishers Inc, New York: VCH, 1988, p. 235.

25. L. M. Prince. A theory of aqueous emulsions. I. Negative interfacial tension at oil/water interface. J. Colloid Interf. Sci. 23(3):165 (1967).

26. Rabolt, J F, Russell, T P, Twieg, R J. Macromolecules 1984, 17, 2786.

27. J. G. Riess. Oxygen carriers (“blood substitutes”)-raison d'etre, chemistry, and some physiology. Chem Rev. 101:2797-2919 (2001).

28. J. G. Riess. Fluorous micro- and nanophases with a biomedical perspective. Tetrahedron 58:4113-4131 (2002).

29. Russell, T P, Rabolt, J F, Twieg, R J, Siemens, R L, Farmer, B L. Macromolecules 1986, 19, 1135.

30. E. Schoof, K. von der Hardt, M. A. Kandler, F. Abendroth, T. Papadopoulos, W. Rascher and J. Dotsch. Aerosolized perfluorocarbon reduces adhesion molecule gene expression and neutrophil sequestration in acute respiratory distress. European Journal of Pharmacology 457(2-3):195-200 (2002).

31. K.-V. Schubert and E. W. Kaler. Microemulsifying fluorinated oil with mixtures of fluorinated and hydrogenated surfactants. Colloid Surface A 84:97-106 (1994).

32. C. Selve, B. Castro, P. Leempoel, G. Mathis, T. Gartiser and J. J. Delpuech. Synthesis of Homogeneous Polyoxyethylene Perfluoroalkyl Surfactants, Tetrahedron 39:1313-1316 (1983).

33. J. Sjoblom, R. Lindberg and S. E. Friberg. Microemulsion-phase equilibria characterization, structures, applications and chemical reactions. Adv. Colloid Interf. Sci. 95:125-258 (1996).

34. Turberg, M P, Brady, J E. J. Am. Chem. Soc. 1988, 110, 7797.

35. Twieg, R J, Russell, T P, Siemens, R L, Rabolt, J F. Macromolecules 1985, 18, 1361.

36. R. M. Winslow. Blood substitutes. Curr. Opin. Hema. 9:146-151 (2002).

1. A fluorosurfactant having a general formula:
R_{F}-L-X or R_{f}-L-X-L-R_{F}, wherein R_{F} is a perfluorocarbon group, X is a hydrophilic moiety, and l is an amide linkage of general structure —CO—NH—.

2. The fluorosurfactant of claim 1, wherein the hydrophilic moiety is selected from the group consisting of polyols, mono- and polysaccharides, polyethylene glycols (PEGs), amino acids, peptides, alginate, amine oxides, phosphoramides, and their derivatives.

3. The fluorosurfactant of claim 1, wherein the hydrophilic moiety is a PEG having a molecular weight from about 200 to about 8000 Daltons or a methoxy-PEG (MPEG) having a molecular weight from about 350 to about 5000 Daltons.

4. The fluorosurfactant of claim 1, wherein the hydrophilic moiety is a PEG having a molecular weight from about 200 to about 1000 Daltons or a MPEG having a molecular weight from about 350 to about 750 Daltons.

5. The fluorosurfactant of claim 1, wherein the perfluorocarbon group has a skeletal chain that has from about 3 to about 17 carbon atoms.

6. The fluorosurfactant of claim 5, wherein the skeletal chain has about 7 carbon atoms.

7. An emulsion comprising a perfluorocarbon (PFC) forming an oil phase, an aqueous solution forming a water phase, and the fluorosurfactant of claim 1.

8. The emulsion of claim 7, wherein the aqueous solution comprises water and at least one additional component selected from a group consisting of buffering agents, salts, water-soluble drugs, gene-containing compositions, and nutrients.

9. The emulsion of claim 7, wherein the aqueous solution is water.

10. The emulsion of claim 7, wherein the PFC comprises perfluoroctyl bromide (PFOB) or perfluoropolyether (PFPE).

11. The emulsion of claim 7, wherein the PFC, the aqueous solution, and fluorosurfactant are mixed in such amounts that a thermodynamically stable and clear microemulsion is obtained.

12. The microemulsion of claim 11, wherein the microemulsion is a PFC-in -water microemulsion or a water-in-PFC microemulsion

13. The microemulsion of claim 11, wherein the microemulsion retains its stability and clarity for at least one month at 25° C.

14. The microemulsion of claim 11, wherein the perfluorocarbon group of the fluorosurfactant and a perfluorocarbon group of the PFC have a similar structure.

15. The microemulsion of claim 11, wherein the perfluorocarbon group of the fluorosurfactant and the perfluorocarbon group of the PFC have a similar molecular weight.

16. A microemulsion comprising a PFC forming an oil phase, water or aqueous solution forming a water phase, and a PEG-based fluorosurfactant with an HFB value from about 7 to about 13, wherein
HFB=(mol % hydrophilic group)/5.

17. The microemulsion of claim 16, wherein the fluorosurfactant has HFB value of about 9.

18. The microemulsion of claim 16, wherein the fluorosurfactant selected from a group consisting of C₇F₁₅CONH-PEG₃₅₀-OCH₃, C₇F₁₅CONH-PEG₅₅₀-OCH₃, and C₇F₁₅CONH-PEG₇₅₀-OCH₃.

19. The microemulsion of claim 16, wherein the fluorosurfactant comprises MPEG having a molecular weight from about 350 to about 750 Daltons.

20. The microemulsion of claim 16, wherein the fluorosurfactant comprises a first perfluorocarbon group and the PFC comprises a second perfluorocarbon group, and wherein the first and the second perfluorocarbon groups have a similar structure.

21. The microemulsion of claim 20, wherein the first and the second perfluorocarbon groups have a similar molecular weight.

22. The microemulsion of claim 16, wherein the PFC is PFOB or PFPE.

23. A method of amidification of a fluorocompound, the method comprising mixing perfluoroacid chloride of a general formula CₙF₂ₙ₊₁COCl with a compound containing an amine group with a general structure R—NH₂ under reaction conditions sufficient to obtain a product having a general formula CₙF₂ₙ₊₁—CONH—R.

24. The method of claim 23 further comprising a step of dissolving perfluoroacid chloride in hexane to obtain a first mixture prior to mixing perfluoroacid chloride with the amino-modified compound.

25. The method of claim 24 further comprising a step of mixing dichloromethane with the amino-modified compound to form a second mixture prior to combining with the first mixture.

26. The method of claim 25, wherein the reaction conditions comprise adding the first reaction mixture by drops to the second reaction mixture.

27. The method of claim 23, wherein the conditions further comprise incubating mixed ingredients at about 4° C. in an ice bath.

28. The method of claim 23, wherein the reaction conditions comprise combining the amino-modified compound and perfluoroacid chloride in an 1:1.1 molar ratio.

29. The method of claim 23, wherein n=6-13.

30. The method of claim 23, wherein the R—NH₂ compound is a MPEG-N H₂.

31. The method of claim 30, wherein MPEG has a molecular weight from about 350 to about 750.

32. The method of claim 30 further comprising steps of:
(a) heating a mixture of MPEG-OH with thionyl chloride and pyridine to obtain MPEG-Cl; and
(b) heating a mixture of MPEG-Cl in ethanol solution saturated with excess ammonia to obtain the MPEG-NH₂.

33. The method of claim 23, wherein the R—NH₂ compound is an amino-alkan.

34. The method of claim 33, wherein the amino-alkan is dodecylamine or octylamine.

35. The method of claim 23 or 33, wherein the conditions comprise room temperature for 30 minutes, followed by 60° C. for 6 hours.

36. The method of claim 23, wherein the R—NH₂ compound is an amino-modified compound or a compound naturally contains an amine group.

37. The method of claim 36, wherein the amino-modified compound is selected from a group consisting of polyols, mono- and polysaccharides, PEGs, amino acids, peptides, alginate, amine oxides, phosphoramides, and their derivatives.

38. A method of preparing PFC-in-water or water-in-PFC emulsions comprising:
(a) calculating HFB value of fluorosurfactants of claim 1 using a formula:
HFB=(mol % hydrophilic group)/5;
(b) selecting a candidate fluorosurfactant with HFB value in the range from about 6 to about 13; and
(c) mixing the selected fluorosurfactant with an aqueous solution or water and PFC.

39. The method of claim 38, wherein the emulsion is a stable and clear microemulsion.

40. The method of claim 38, wherein step (b) further comprises a step of selecting a fluorosurfactant with CₙF₂ₙ₊₁ group, which has a molecular weight, structure, or both molecular weight and structure similar to that of the PFC.

41. A perfluoroalkanamide of a general structure R_{F}-L-RH, wherein R_{F} is a perfluorocarbon group, L is an amide linkage of general structure —CO—NH—, and R_{H═C}ₘH₂ₘ₊₁.
